(19) United States
(12) Patent Application Publication
(10) Pub. No.: US 2021/0290833 A1
(43) Pub. Date: Sep. 23, 2021

(54) METHOD, MICROCHANNEL STRUCTURE AND MICROCHANNEL SYSTEM FOR REMOVING CIRCULATING TUMOR CELLS IN BLOOD

(71) Applicant: LifeCode Biotech, Taipei City (TW)

(72) Inventors: Joe-Yuan DOONG, Chiayi (TW); Sung-Chi TSAI, Chiayi (TW)

(21) Appl. No.: 16/823,431

(22) Filed: Mar. 19, 2020

(51) Int. Cl. A61M 1/36; A61M 1/38

(52) U.S. Cl. CPC A61M 1/3621; A61M 1/361; A61M 1/38

(57) ABSTRACT

The present invention provides a microchannel structure for removing circulating tumor cells in a circulating blood system without damaging cells in the blood, wherein the microchannel is loaded with a plurality of beads. The microchannel structure includes: a blood sample entrance passing a blood sample therethrough; a bead mooring section including: a first end connected to the blood sample entrance; a second end; a first section being relatively close to the first end, and cooperating with the first end to cause the plurality of beads to form a bead array in the bead mooring section for decreasing a flow rate of the blood sample through an interstice among neighboring ones of the plurality of beads; and a second section being relatively close to the second end, and causing the treated blood sample to smoothly flow therethrough; and a blood sample exit connected to the second end. One of the applications of this invention is to remove cancer cells in cancer patient's circulating blood system.

### FIELD OF THE INVENTION

The present invention is related to a method, a microchannel system, a microchannel chip, and a microchannel structure for removing the circulating tumor cells in blood. Particularly, the present invention is related to a method, a microchannel system, a microchannel chip, and a microchannel structure for removing the circulating tumor cells in blood without damaging blood cells.

### BACKGROUND OF THE INVENTION

The high mortality rate caused by cancer has been a serious problem in the health care field for a long time. Studies have found that tumors in early development stage are mostly an organ-constrained disease. However, tumors always spread from the primary site to a distant organ through the blood and form new tumors in secondary sites, a phenomenon known as metastasis. Such distant metastasis is the main cause of death in cancer patients. Cells that fall off the primary site of the tumor and enter the blood circulation system are called circulating tumor cells (CTCs). CTCs are considered a necessary prerequisite for the occurrence of the distal tumor metastasis. The accurate counting of CTCs and molecular biomarkers are important indicators for the prognosis of cancer patients, and to judge and evaluate the effectiveness of the treatment.

However, in the prior art, a device for detecting and collecting circulating tumor cells has the disadvantages of a low detection rate and a low collection purity, and the tested sample cannot be reused (such as transfusing the filtrated sample back into an individual). Therefore, there is an urgent need for a low cost, high sensitivity, high specificity, high efficiency, and convenient system and method not only for CTC detection but also for removing circulating tumor cells in the blood.

It is therefore the Applicant's attempt to deal with the above situation and shortcomings encountered in the prior art.

### SUMMARY OF THE INVENTION

The present invention provides a microchannel chip that can detect and remove rare cells in the blood. The microchannel chip uses transparent beads moored therein to form a special bead array to decrease the flow rate of the blood. The microchannel chip can catch the rare cells circulating in the blood more efficiently and without damaging the normal heathy blood cells, especially the cells which are carcinogenic or cancerous, such as circulating tumor cells, can be removed from the blood. The remaining blood, after removal of the rare cells, can be collected, pooled and transfused back to the subject. The method and device described in the present invention can be used in an adjuvant cancer therapy, a blood bag dialysis, immunotherapy and other treatments for metastatic cancers.

In accordance with another aspect of the present disclosure, a microchannel system for removing circulating tumor cells in a circulating blood system without damaging red or white blood cells in the blood is disclosed. The microchannel system includes: a sample collecting area for collecting therefrom a blood sample to be treated; a microchannel chip connected to the sample collecting area, and including a microchannel structure having: a blood sample entrance passing the blood sample therethrough; a bead mooring section having a first end connected to the blood sample entrance, and a second end; a bead blocking wall configured in the bead mooring section, being relatively close to the second end, and causing a plurality of beads to be moored in the bead mooring section and to form a bead array in the bead mooring section to decrease a flow rate of the blood sample in the bead mooring section; and a blood sample exit connected to the second end; and a pump connected to the microchannel chip, and generating a negative pressure to cause the blood sample to flow through the microchannel structure.

In accordance with one more aspect of the present disclosure, a microchannel structure for removing circulating tumor cells in a circulating blood system without damaging cells in the blood, wherein the microchannel is loaded with a plurality of beads is disclosed. The microchannel structure includes: a blood sample entrance passing a blood sample therethrough; a bead mooring section including: a first end connected to the blood sample entrance; a second end; a first section being relatively close to the first end, and cooperating with the first end to cause the plurality of beads to form a bead array in the bead mooring section for decreasing a flow rate of the blood sample through an interstice among neighboring ones of the plurality of beads; and a second section being relatively close to the second end, and causing the treated blood sample to smoothly flow therethrough; and a blood sample exit connected to the second end.

In accordance with one more aspect of the present disclosure, a method for removing circulating tumor cells in a circulating blood system without damaging cells in the blood is provided. The method includes: providing a microchannel structure mooring therein a plurality of beads formed as a bead array to decrease therein a flow rate of the blood, wherein each of the plurality of beads has a surface coated therewith a plurality of antibodies; obtaining a blood sample from a subject; causing the blood sample to flow through the microchannel structure; and capturing circulating tumor cells in the blood sample with multiple antibodies.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other objectives, advantages and efficacies of the present invention will be described in detail below taken from the preferred embodiments with reference to the accompanying drawings.

FIG. 1 shows a schematic diagram of a microchannel system of the present invention.

FIG. 2 shows a top schematic diagram of a microchannel chip of the present invention.

FIG. 3 shows a sectional schematic diagram of a cut view of the microchannel chip along the section line A-A′ in FIG. 2.

FIG. 4 shows a top schematic diagram of a bead mooring section without the plurality of beads of the present invention.

FIG. 5 shows a top schematic diagram of a bead mooring section with the plurality of beads forming a bead array of the present invention.

FIG. 6 shows a perspective diagram of a bead mooring section with the plurality of beads forming a bead array of the present invention.

FIG. 7 shows a flowchart of a method for removing circulating tumor cells in a blood using the microchannel chip of the present invention.

FIG. 8 shows another embodiment of the microchannel chip of the present invention.

FIG. 9 shows a diagram of the results of the catching rate of the circulating tumor cells and the survival rate of the blood cells using the microchannel system of the present invention under different negative pressures.

FIG. 10 shows a diagram of the effect of different concentrations of the antibodies on the beads for the catching rate of the circulating tumor cells.

FIGS. 11(A)˜11(C) show image diagrams of the stained blood specimen flowing through the microchannel system of the present invention, wherein FIG. 11(A) is an image diagram under an optical microscope without fluorescence, and FIGS. 11(B)˜11(C) are image diagrams under a fluorescence microscope.

### DETAILED DESCRIPTION OF THE PREFERRED EMBODIMENTS

The present invention will now be described more specifically with reference to the following embodiments. It is to be noted that the following descriptions of preferred embodiments of this invention are presented herein for purpose of illustration and description only; they are not intended to be exhaustive or to be limited to the precise form disclosed. In the preferred embodiments, the same reference numeral represents the same element in each embodiment.

In any embodiment of the present invention, each section of the microchannel structure includes an upper wall, a bottom wall, a left side wall and a right side wall. The aperture of each section of the microchannel structure includes a width and a depth, wherein the width represents a distance from the left side wall to the right side wall, and the depth represents a distance from the upper wall to the bottom wall.

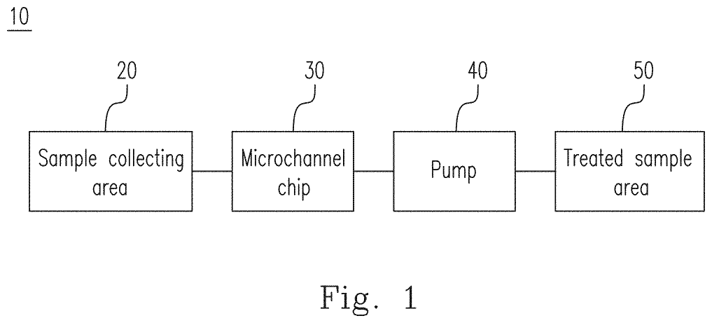

Please refer to FIG. 1, which is a schematic diagram of a microchannel system of the present invention. The microchannel system 10 of the present invention includes a sample collecting area 20, a microchannel chip 30 configure a plurality of beads therein, a, pump 40 and a treated sample area 50. The sample collecting area 20 is used for collecting and providing a blood sample to be treated. The sample collecting area 20 can be a blood sample storage device, such as a blood collection tube, a blood bag or a tube connecting to a blood vessel of a human body, and guides the blood sample to the microchannel chip 30. The microchannel chip 30 connects to the sample collecting area 20, and the microchannel chip 30 is used to remove circulating tumor cells in a blood without damaging blood cells in the blood. The treated sample area 50 is used to recover the treated blood sample. The treated sample area 50 can be a blood sample storage device, such as a blood collection tube, a blood bag or a tube connecting to a blood vessel of human body, and guides the treated blood sample to the blood vessel of human body. The pump 40 generates a negative pressure and maintains the sample collecting area 20 and the microchannel chip 30 at a negative pressure state to cause the blood sample to flow through the microchannel chip 30. The pump 40 of the present invention can be configured between the microchannel chip 30 and the treated sample area 50, so that the treated blood sample flows through the pump 40 first and then to the treated sample area 50 after flowing out of the microchannel chip 30. The pump 40 of the present invention can be also configured after the treated sample area 50, so that the treated blood sample flows to the treated sample area 50 directly after flowing out of the microchannel chip 30. The pump 40 of the present invention can be a pump which can cause the microchannel chip 30 to be under negative pressure, such as an air extracting pump, a vacuum pump, or a peristaltic pump.

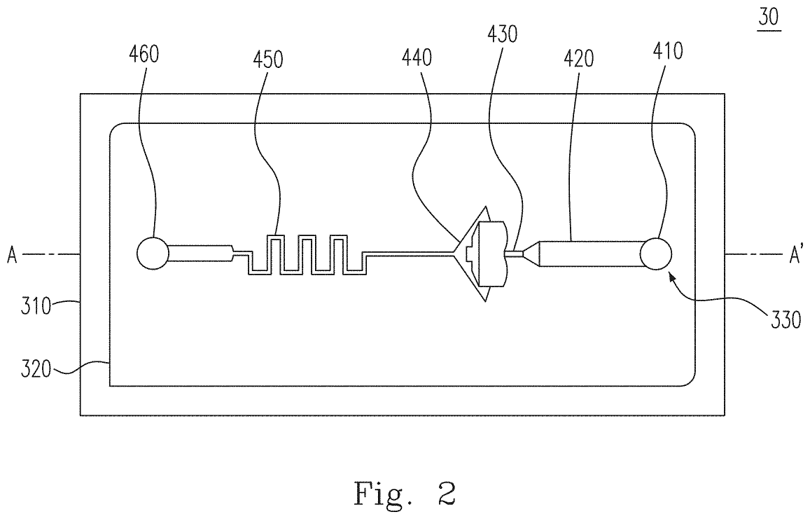

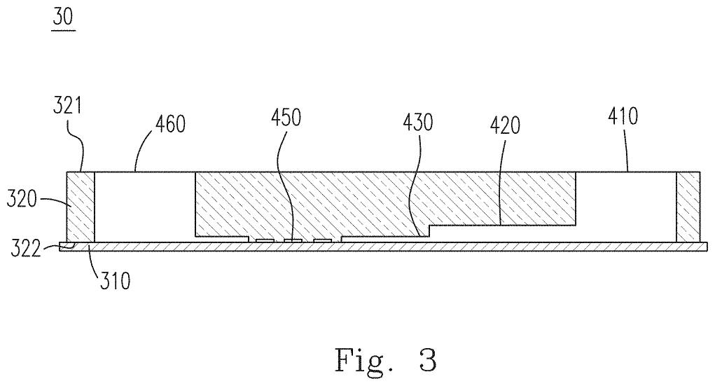

Please refer to FIGS. 2-3, which are top schematic diagrams and sectional schematic diagrams of the microchannel chip of the present invention. The microchannel chip 30 of the present invention includes a substrate 310, a body 320, and a microchannel structure 330. The body 320 has a first surface 321 and a second surface 322 opposite to the first surface 321, and the second surface 322 covers the substrate 310. The microchannel structure 330 is formed on the second surface 322 of the body 320 to form a microchannel between the body 320 and the substrate 310.

The microchannel structure 330, from the entrance to the exit, sequentially includes a blood sample entrance 410, an expanding section 420, a resistance-increasing section 430, a bead mooring section 440, a slow flow section 450 and a blood sample exit 460. The blood sample entrance 410 of the present invention extends from the first surface 321 to the second surface 322 of the body 320 to pass a blood sample therethrough. The blood sample entrance 410 can connect to a blood collection tube, a blood bag or a tube connecting to a blood vessel of human body. The blood sample entrance 410 can be a circular or a polygonal aperture, preferably the circular aperture. The blood sample entrance 410 has a diameter of 1.5 mm.

The expanding section 420 of the present invention communicates with the blood sample entrance 410. The expanding section 420 has an aperture which can be a circle or a polygon, preferably a square. The expanding section 420 can cushion the rapid flow of the blood sample to increase a unit flow (volume passed per second) of the expanding section 420, and prevent the blood sample from leaking due to excessive hydraulic pressure. The width of the expanding section 420 of the present invention is 1.5 mm, and a depth of the expanding section 420 sets between 0.2˜1.5 mm.

The resistance-increasing section 430 of the present invention communicates with the expanding section 420. The resistance-increasing section 430 has an aperture which can be a circle or a polygon, preferably a square. The width of the resistance-increasing section 430 is smaller than the width of the bead mooring section 440, the expanding section 420 and the diameter of the blood sample entrance 410. Therefore, the resistance-increasing section 430 can enhance a fluid resistance to decrease the flow rate of the blood sample, which has the function of preventing the blood sample from bursting and limiting the flow of the blood sample. The width of the resistance-increasing section 430 of the present invention is 0.3 mm, and the depth of the resistance-increasing section 430 is 0.2 mm.

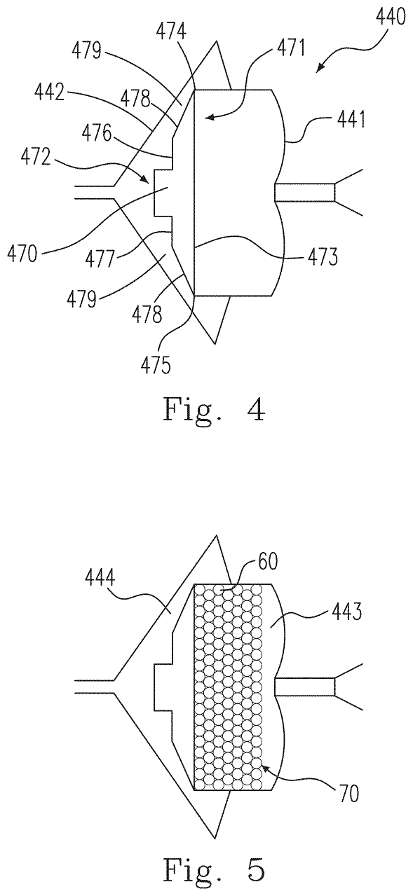

Please refer to FIGS. 4-5, which are top schematic diagrams of the bead mooring section with and without the plurality of beads of the present invention. The bead mooring section 440 of the present invention has a first end 441 and a second end 442, wherein the first end 441 connects to the resistance-increasing section 430, and the second end 442 connects to the slow flow section 450. A plurality of beads 60 are moored in the bead mooring section 440, as shown in FIG. 5.

The first end 441 of the bead mooring section 440 can be formed in various shapes, such as linear shape, curved shape and multi-curved shape, preferably curved. The curved shape at the first end 441 causes the blood sample to be dispersedly flowed through an interstice among neighboring ones of the plurality of beads 60.

A bead blocking structure 470 is configured in the bead mooring section 440 and close to the second end 442. The bead blocking structure 470 includes an inlet side 471 and a centrally protruding outlet side 472, wherein the inlet side 471 is a side closer to the first end 441 of the bead mooring section 440 than the centrally protruding outlet side 472, and the centrally protruding outlet side 472 is a side closer to the second end 442 of the bead mooring section 440 than the inlet side 471. The inlet side 471 is a bead blocking wall 473 causing the plurality of beads 60 to be moored in the bead mooring section 440 and to form a bead array 70 between the bead blocking wall 473 and the first end 441 of the bead mooring section 440. The blood sample is treated by the bead array 70. The bead array 70 can decrease the flow rate of the blood sample through interstices among neighboring ones of the plurality of beads 60 in the bead mooring section 440. The bead blocking wall 473 is a first section 443 in the bead mooring section 440, as shown in FIG. 5. The bead blocking wall 473 has a first side end 474 and a second side end 475, the centrally protruding outlet side 472 has a first outlet side 476 and a second outlet end 477, and the first side end 474 extends to the first outlet side 476 and the second side end 475 extends to the second outlet end 477 to respectively form a inclined surface 478. Two channels 479 are each formed between the second end 442 and each of the two inclined surfaces 478 to cause the treated blood sample to smoothly flow therethrough and flow to the slow flow section 450. In an embodiment, the centrally protruding outlet side 472 is a stepped part. The centrally protruding outlet side 472 is a second section 444 in the bead mooring section 440, as shown in FIG. 5.

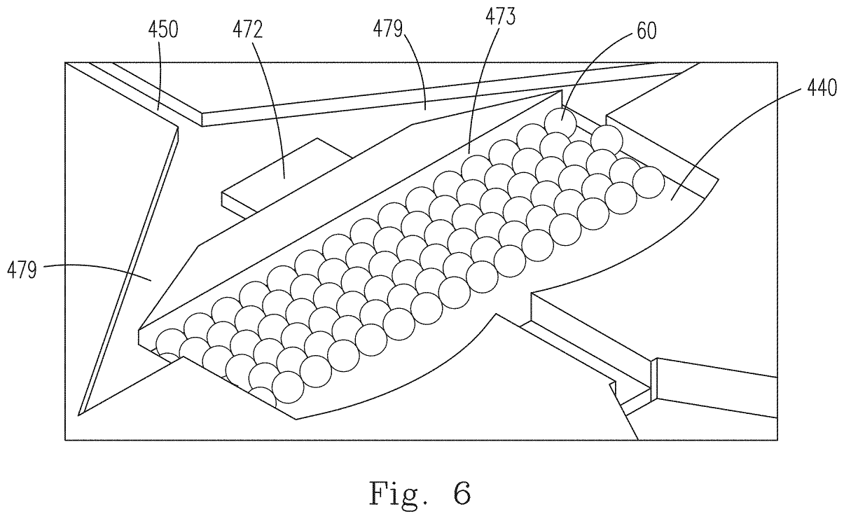

The width of the bead mooring section 440 in the present invention affects the flow rate of the blood sample in the microchannel structure 330. Different flow rates affect the catching rate of the circulating tumor cells. In the present invention, the larger the width of the bead mooring section 440, the better the catching rate of the circulating tumor cells. In the present invention, the depth of the bead mooring section 440 is larger than the particle size of beads 60. In an embodiment, the width of the bead mooring section 440 sets between 0.3˜4.8 mm, and the depth of the bead mooring section 440 sets between 0.08˜1.5 mm. For mooring the beads 60 in the bead mooring section 440 to form the bead array 70, the aperture of the two channels 479 are smaller than the particle size of the beads 60 (as shown in FIG. 6) to prevent the beads from flowing into the two channels 479. In an embodiment, the depth of the two channels 479 is less than 0.05 mm.

100-500 beads 60 can be accommodated in the bead mooring section 440 of the present invention to form the bead array 70. The bead array 70 can effectively cause the flow rate of the blood sample to flow uniformly or slowly among the interstices among neighboring ones of the beads 60, which can decrease the damage of the blood cells in the blood sample and significantly increase the catching rate of the circulating tumor cells. According to Bernoulli's principle, the flow rate of a blood sample will increase when the blood sample flows through the interstice. The increase of the flow rate of the blood sample will cause the blood cells in the blood sample to be damaged, and cause the contact time between the blood sample and the beads to be decreased. The two channels 479 in the bead mooring section 440 can provide multiple routes for the blood sample flowing among the bead array 70 to achieve an effect of uniform flow or slow flow, so as to prevent the blood sample from accelerating while flowing through the interstice.

The particle size of each bead set between 10˜200 pin. A material of the beads is a plastic material having biocompatibility. Bioactive components are coated on a surface of each bead. The bioactive components can be any components that can catch rare cells, including antibodies, aptamers, short-chain peptides or saccharides. In the present invention, the bioactive component binds biotin, each bead binds streptavidin, and the biotin on the biological component will bind to the streptavidin on the bead, so that the reactive substance of the biological component on the bead will face toward the outside to catch the rare cell in the blood more effectively. For example, when the biological component is an antibody, the biotin will bind to the bottom of the heavy chain, i.e. carboxyl end of the heavy chain. Hence, after the biotin on the antibody binds to the streptavidin on the bead, an end of the antibody having the reactive substance (such as EpCAM) will face toward the blood sample to catch the rare cell in the blood sample with the greatest probability.

Please refer to FIGS. 2-3, the slow flow section 450 of the present invention connects to the second end 442 of the bead mooring section 440. The slow flow section 450 has an aperture which can be a circle or a polygon, preferably a square. The aperture of the slow flow section 450 is less than the aperture of the bead mooring section 440, the particle size of the bead 60 and, and the slow flow section 450 has a labyrinth structure to decrease and stabilize the flow rate of the blood sample in the microchannel structure 330. The width of the slow flow section 450 of the present invention is 0.2 mm, and the depth of the slow flow section 450 is less than 0.05 mm.

The blood sample exit 460 of the present invention connects to the slow flow section 450, and extends from the second surface 322 to the first surface 321 of the body 320. The treated blood sample passing through the microchannel structure 330 will flow to the pump 40 or the treated sample area 50 through the blood sample exit 360. The treated blood sample can be transfused back to the subject. The blood sample exit 460 can be a circular or a polygonal aperture, preferably the circular aperture. The blood sample exit 460 has a diameter of 1.5 mm.

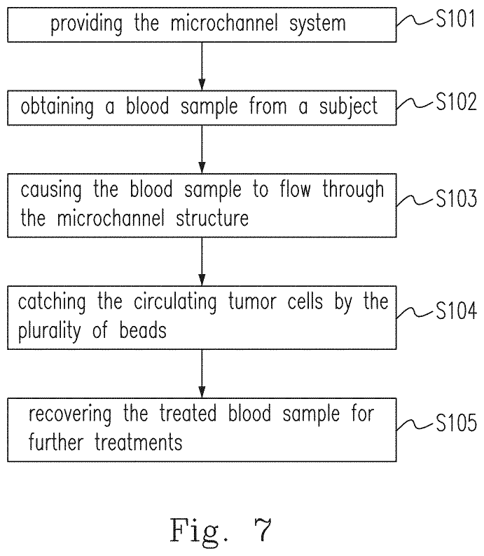

Please refer to FIGS. 1-5 and 7, the method for removing circulating tumor cells in a blood without damaging blood cells in the blood of the present invention includes: providing the microchannel system 10, wherein the microchannel system 10 includes the microchannel structure 330 mooring therein a plurality of beads 60, and the plurality of beads 60 forms a bead array 70 (Step S101); obtaining a blood sample from a subject (Step S102); causing the sample to flow through the microchannel structure 330 (Step S103); catching the circulating tumor cells in the blood sample by the plurality of beads 60 (Step S104); and recovering the treated blood sample for further processes (Step S105). In Step S101, a plurality of antibodies are coated on a surface of each of the plurality of beads 60, and the plurality of antibodies catch the circulating tumor cells in the blood sample. In Step S102, the sample can be obtained from an organism, preferably blood from a human body, using a blood collection tube, a blood bag or a tube connecting to a blood vessel. In Steps S103˜105, after connecting the blood collection tube, the blood bag or the tube to the microchannel chip 30, the blood sample flows through the blood sample entrance 410, the expanding section 420, the resistance-increasing section 430, the bead mooring section 440, the slow flow section 450 and the blood sample exit 460 (Step S103), wherein the antibodies on the surface of the beads catch the circulating tumor cells in the blood sample at the bead mooring section 440 (Step S104), so that the circulating tumor cells isolated from the blood sample is located in the bead mooring section 440, and the treated blood sample flows to another clean blood collection tube, blood bag or tube after flowing out of the blood sample exit 360 (Step S105) for further treatments.

The amount of circulating tumor cells in the treated blood sample is less than that in the untreated blood sample and the blood cells are not damaged in the treated blood sample after passing through the microchannel chip of the present invention. Therefore, the microchannel system and the method thereof can be used as an adjuvant treatment for cancer and a treatment for metastatic cancer. That is to say, the treated blood sample can be transfused back to the original subject. The method of the present invention can be implemented multiple times until there is no circulating tumor cell in the subject.

In another embodiment of the method of the present invention, the treated blood sample can be also transfused to another subject in need thereof. Therefore, in the treated blood sample it can be detected whether there still are the circulating tumor cells in the treated blood sample. If there are no circulating tumor cells in the treated blood sample, the treated blood sample can be transfused to another subject in need thereof.

The circulating tumor cells located in the bead mooring section 440 can be observed directly using a microscope, and/or washed out by known techniques for further experiments and analysis.

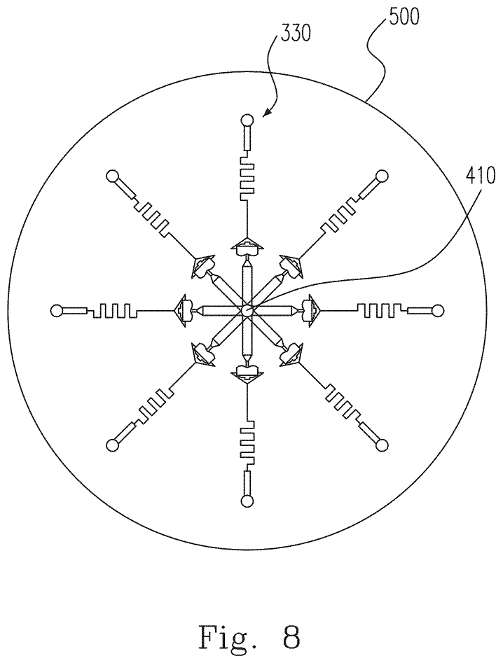

In another embodiment of the present invention, a plurality of the microchannel structures 330 can be combined to form a microchannel chip 500, as shown in FIG. 8. The microchannel chip 500 can be any shape, preferably a disc shape. In an embodiment of the disc shape, each of the microchannel structure 330 is disposed in the microchannel chip 500 in a radial form. Specifically, the plurality of the microchannel structures 330 share the same blood sample entrance 410. The blood sample entrance 410 is located at or near the center of the microchannel chip 500, and the remaining sections of the microchannel structure 330 are gradually disposed toward the periphery of the microchannel chip 500. In the microchannel chip 500 of the present invention, when the blood sample is injected from the blood sample entrance 410, the blood sample will evenly flow into every microchannel structure 330, causing a great amount of the blood sample to be analyzed in a short time to achieve a concept of high-throughput. Because the circulating tumor cell is generally low in the blood in the early tumor period, the concept of high-throughput was added to the microchannel chip in the whole blood test, so that the blood can be analyzed in a greater amount than using only one microchannel structure in a specific time. In an embodiment, the microchannel chip 500 has a radius of 4 cm, and a number of the microchannel structure 330 in the microchannel chip 500 is 2˜8.

The material of the substrate 310 in the present invention can be polymethylmethacrylate (PMMA), polyethylene terephthalate (PET), polycarbonate (PC), polydimethylsilicon (PDMS), silica gel, rubber, plastic or glass. The material of the body 320 can be polymethylmethacrylate (PMMA), polyethylene terephthalate (PET), polycarbonate (PC), polydimethylsilicon (PDMS), silica gel, rubber or plastic. The material property between the substrate 310 and the body 320 should be considered when choosing the materials of the substrate 310 and the body 320. The substrate 310 and the body 320 are transparent, and thus the microchannel chip 30 can be observed by any optical device.

The manufacturing method of the microchannel chip of the present invention includes: printing a master mold having a microchannel structure using a 3D printer, wherein the master mold is a light-cured resin washed by 95% alcohol; curing the master mold by UV light for 2 minutes; after washing by alcohol, baking the master mold for 10 minutes; pouring a food-grade material PDMS into the master mold; curing at 80° C. for 50 minutes to obtain a body having the microchannel structure; and jointing the body with a glass substrate using an oxygen plasma machine to obtain the microchannel chip. Finally, the beads coated with biological components are loaded into the microchannel chip, the blood sample entrance is connected to the sample collecting area, and the blood sample exit is connected to the treated sample area to obtain the microchannel system of the present invention.

### Experiment Example

The Relationship Between the Negative Pressure Condition, and Circulating Tumor Cells Catching Rate and Blood Cells Survival Rate

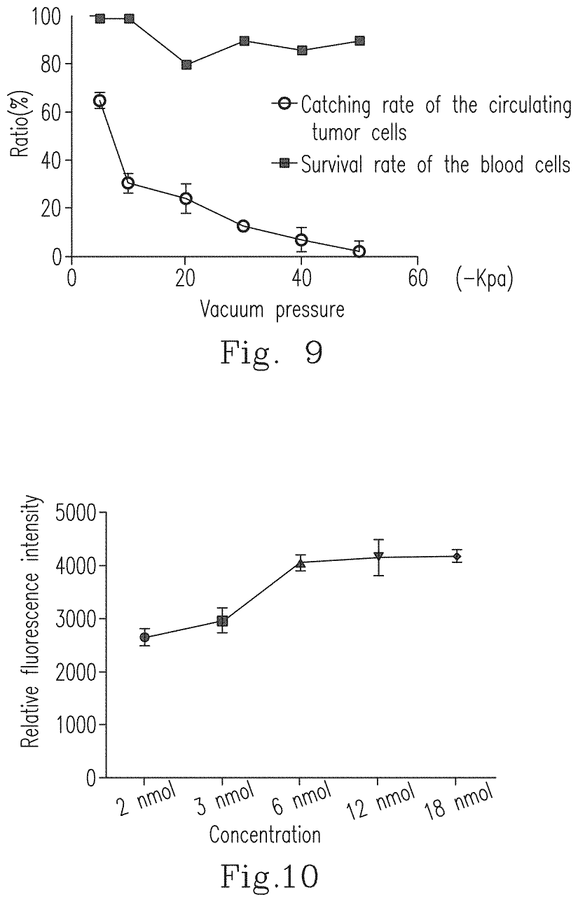

The vacuum pump is used in the present invention to make the microchannel structure in the negative pressure state, so that the blood sample flows in the microchannel chip for catching the circulating tumor cells by the beads. Different vacuums relate to the circulating tumor cells catching rate and the blood cells survival rate. The higher the vacuum, the higher the negative pressure in the microchannel structure, causing the blood sample to flow faster in the microchannel structure. Please refer to FIG. 9, which is a diagram of the results of the catching rate of the circulating tumor cells and the survival rate of the blood cells using the microchannel system of the present invention under different negative pressures. In FIG. 9, the results show that the catching rate of the circulating tumor cells at low negative pressure is higher than that at high negative pressure, and the survival rate of the blood cells at low negative pressure is higher than that at high negative pressure. In addition, FIG. 9 shows that there is the best catching rate and the highest survival rate at negative pressure state between 5˜10-Kpa. Therefore, the survival rate of the blood cells is higher and the catching rate of the circulating tumor cells is better when the flow rate is lower.

Detecting Sensitivity of the Microchannel System

Different numbers (10, 20, 30, 40, 50 and 100) of spiked cancer cells are respectively added into different tubes having 2000 μL blood to observe the detecting sensitivity of the microchannel system and the survival rate of the blood cells, and the result is shown in Table 1. In Table 1, it can be seen that even when there are only 10 spiked cancer cells in blood, the microchannel system of the present invention still can catch the spiked cancer cells and the survival rate of the blood cells is 100%. Therefore, the detecting sensitivity of the microchannel system of the present invention is very high without damaging the blood cells.

TABLE 1

| Total volume | Amount | Catching amount | Survival rate |
|---|---|---|---|
| of the blood | of the spiked | of the cancel | of the blood |
| sample | cancer cells | cells | cells |
| 2000 (μL) | 100 | 72 ± 10 | 100% |
| | 50 | 30 ± 8 | 100% |
| | 40 | 21 ± 8 | 100% |
| | 30 | 12 ± 7 | 100% |
| | 20 | 3 ± 1 | 100% |
| | 10 | 2 ± 1 | 100% |

Relationship Between Concentration of Antibodies and Catching Rate of Circulating Tumor Cells

Different concentrations (2 nmol, 3 nmol, 6 nmol, 12 nmol and 18 nmol) of the fluorescence-labeled antibodies are coated on the beads, followed by loading the beads into the microchannel chip to observe the effect of different concentrations of the antibodies on the beads for the catching rate of the circulating tumor cells, and the result is shown in FIG. 10. In FIG. 10, x-axis is the concentration of the antibodies, and y-axis is the fluorescence intensity relative to no fluorescence. It can be seen in FIG. 10 that when the concentration of the antibodies is 2 nmol, the beads can effectively catch the circulating tumor cells, and when the concentration of the antibodies is 6 nmol, the effect of the beads for catching the circulating tumor cells is the best. Therefore, the concentration of the antibodies on the beads for catching the circulating tumor cells is between 2˜6 nmol.

Actual Detecting Result of the Microchannel System

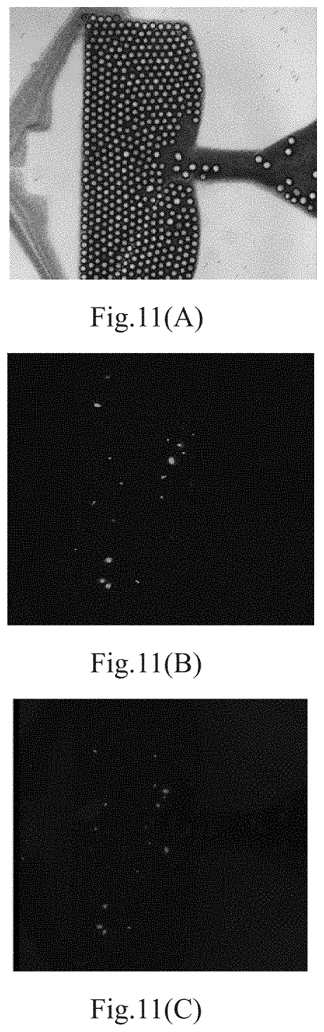

The blood specimen having MCF-7 cancer cells was stained using a CaAM (Calcein Acetoxymethylester) fluorescent dye and a Hoechst fluorescent dye, followed by flowing through the microchannel system of the present invention, and the results are shown in FIGS. 11(A)˜11(C). FIG. 11(A) shows an image diagram under a generally optical microscope, wherein the bead mooring section is observed. FIG. 11(B) shows that the positions of the MCF-7 cancer cells (green fluorescence positions) caught by the beads. FIG. 11(C) shows that the positions of DNA of the MCF-7 cancer cells (blue fluorescence positions) stained by the Hoechst fluorescent dye. In can be seen in FIGS. 11(A)˜11(C) that the positions of green fluorescence are identical to those of blue fluorescence, and hence, it can be confirmed that the beads can catch the cancer cells after the blood specimen flows through the special bead array.

### Embodiments

1. A microchannel system for removing circulating tumor cells in a blood without damaging blood cells in the blood, including: a sample collecting area for collecting therefrom a blood sample to be treated; a microchannel chip connected to the sample collecting area, and including a microchannel structure having: a blood sample entrance passing the blood sample therethrough; a bead mooring section having a first end connected to the blood sample entrance, and a second end; a bead blocking wall configured in the bead mooring section, being relatively close to the second end, and causing a plurality of beads to be moored in the bead mooring section and to form a bead array in the bead mooring section to decrease a flow rate of the blood sample in the bead mooring section; and a blood sample exit connected to the second end; and a pump connected to the microchannel chip, and generating a negative pressure to cause the blood sample to flow through the microchannel structure.

2. The microchannel system according to Embodiment 1, wherein the microchannel structure further includes two channels each formed between the bead mooring section and each end of the bead blocking wall.

3. The microchannel system according to Embodiment 1 or 2, wherein each of the plurality of beads has a particle size, each of the two channels has an aperture, and the particle size is larger than the aperture to prevent the plurality of beads from flowing into the two channels.

4. The microchannel system according to any one of Embodiments 1 to 3, wherein the microchannel structure further includes a bead blocking structure having: an inlet side being relatively close to the first end, having a first side end and a second side end, and being the bead blocking wall; a centrally protruding outlet side being relatively close to the second end, and having a first outlet side and a second outlet side; and two inclined surfaces respectively extended from the first inlet side and the second inlet side to the first outlet side and the second outlet side, wherein the two channels are each formed between the second end and each of the two inclined surfaces to cause the treated blood sample to smoothly flow therethrough.

5. The microchannel system according to any one of Embodiments 1 to 4, wherein each of the plurality of beads includes a surface having a plurality of antibodies to catch the tumor cells circulated in the blood sample.

6. The microchannel system according to any one of Embodiments 1 to 5, wherein the microchannel structure further includes: a resistance-increasing section configured between the blood sample entrance and the first end; and a slow flow section configured between the blood sample exit and the second end, wherein the resistance-increasing section and the slow flow section decrease the flow rate of the blood sample in the bead mooring section.

7. The microchannel system according to any one of Embodiments 1 to 6, further including a treated sample area connected to the blood sample exit or the pump, wherein the treated sample area recovers the treated blood sample.

8. The microchannel system according to any one of Embodiments 1 to 7, wherein the pump is an air extracting pump, a vacuum pump, or a peristaltic pump.

9. A microchannel structure for removing circulating tumor cells in a blood without damaging cells in the blood, wherein the microchannel is loaded with a plurality of beads, and includes: a blood sample entrance passing a blood sample therethrough; a bead mooring section including: a first end connected to the blood sample entrance; a second end; a first section being relatively close to the first end, and cooperating with the first end to cause the plurality of beads to form a bead array in the bead mooring section for decreasing a flow rate of the blood sample through an interstice among neighboring ones of the plurality of beads; and a second section being relatively close to the second end, and causing the treated blood sample to smoothly flow therethrough; and a blood sample exit connected to the second end.

10. The microchannel structure according to Embodiment 9, wherein the first section is a bead blocking wall built in the bead mooring section, the first end has a curvy structure, the plurality of beads are blocked by the bead blocking wall and form the bead array between the bead blocking wall and the first end, and the blood sample is treated by the bead array.

11. The microchannel structure according to Embodiment 9 or 10, wherein the second section has a centrally protruding structure connected to the bead blocking wall, the two sides of the centrally protruding structure faun two channels with the second end, and the two channels cause the treated blood sample to smoothly flow therethrough.

12. The microchannel structure according to any one of Embodiments 9 to 11, wherein the centrally protruding structure is a stepped part.

13. The microchannel structure according to any one of Embodiments 9 to 12, wherein each of the plurality of beads has a particle size, each of the two channels has an aperture, and the particle size is larger than the aperture to prevent the plurality of beads from entering into the two channels.

14. The microchannel structure according to any one of Embodiments 9 to 13, further including a resistance-increasing section configured between the blood sample entrance and the first end, wherein a width of the resistance-increasing section is smaller than that of the bead mooring section, so as to decrease the flow rate of the blood sample in the bead mooring section.

15. The microchannel structure according to any one of Embodiments 9 to 14, further including a slow flow section having a first aperture and configured between the blood sample exit and the second end, wherein the slow flow section is a labyrinth structure, the bead mooring section has a second aperture, and the first aperture is smaller than the second aperture so as to decrease the flow rate of the blood sample in the bead mooring section.

16. A method for removing circulating tumor cells in blood without damaging cells in the blood, including steps of: (a) providing a microchannel structure mooring therein a plurality of beads formed as a bead array to decrease therein a flow rate of the blood, wherein each of the plurality of beads has a surface coated therewith a plurality of antibodies; (b) obtaining a blood sample from a subject; (c) causing the blood sample to flow through the microchannel structure; and (d) catching the circulating tumor cells in the blood sample by the plurality of antibodies.

17. The method according to Embodiment 16, further including steps of: (e) recovering the treated blood sample, wherein an amount of the circulating tumor cells in the treated blood sample is less than that in the untreated blood sample; and (f) transfusing the treated blood sample back to the subject.

18. The method according to Embodiment 16 or 17, further including a step of: (g) repeating step (a) to step (f) until there is no circulating tumor cells in the subject.

19. The method according to any one of Embodiments 16 to 18, further including steps of: (e) recovering the treated blood sample; (f) determining whether there still are the circulating tumor cells in the treated blood sample; and (g) transfusing the treated blood to another subject in need thereof if no circulating tumor cells are detected.

20. The method according to any one of Embodiments 16 to 19, further including a step of: (e) analyzing the circulating tumor cells caught by the plurality of beads.

In summary, the beads coated with the biological components that can identify circulating tumor cells are loaded in the microchannel structure, and form a bead array in the microchannel structure. The microchannel chip having the bead array can increase the sensitivity of the microchannel chip and efficiency of the catching rate without damaging the blood cells, causing the survival rate of the blood cells to be increased. The treated blood sample that had removed the circulating tumor cells has many uses, for example, the treated blood sample can be transfused back to the subject. Because the microchannel chip of the present invention can remove the circulating tumor cells in the blood, the microchannel chip can be used in many ways such as an adjuvant cancer treatment, a blood bag dialysis, a blood immunotherapy and a metastatic cancer cell treatment.

Although the present invention has been described with reference to certain exemplary embodiments thereof, it can be understood by those skilled in the art that a variety of modifications and variations may be made to the present invention without departing from the spirit or scope of the present invention defined in the appended claims, and their equivalents.

What is claimed is:

1. A microchannel system for removing circulating tumor cells in a blood without damaging blood cells in the blood, comprising:
a sample collecting area for collecting therefrom a blood sample to be treated;
a microchannel chip connected to the sample collecting area, and including a microchannel structure having:
a blood sample entrance passing the blood sample therethrough;
a bead mooring section having a first end connected to the blood sample entrance, and a second end;
a bead blocking wall configured in the bead mooring section, being relatively close to the second end, and causing a plurality of beads to be moored in the bead mooring section and to form a bead array in the bead mooring section to decrease a flow rate of the blood sample in the bead mooring section; and
a blood sample exit connected to the second end; and
a pump connected to the microchannel chip, and generating a negative pressure to cause the blood sample to flow through the microchannel structure.

2. The microchannel system as claimed in claim 1, wherein the microchannel structure further includes two channels each formed between the bead mooring section and each end of the bead blocking wall.

3. The microchannel system as claimed in claim 2, wherein each of the plurality of beads has a particle size, each of the two channels has an aperture, and the particle size is larger than the aperture to prevent the plurality of beads from flowing into the two channels.

4. The microchannel system as claimed in claim 2, wherein the microchannel structure further includes a bead blocking structure having:
an inlet side being relatively close to the first end, having a first side end and a second side end, and being the bead blocking wall;
a centrally protruding outlet side being relatively close to the second end, and having a first outlet side and a second outlet side; and
two inclined surfaces respectively extended from the first inlet side and the second inlet side to the first outlet side and the second outlet side, wherein the two channels are each formed between the second end and each of the two inclined surfaces to cause the treated blood sample to smoothly flow therethrough.

5. The microchannel system as claimed in claim 1, wherein each of the plurality of beads includes a surface having a plurality of antibodies to catch the tumor cells circulated in the blood sample.

6. The microchannel system as claimed in claim 1, wherein the microchannel structure further includes:
a resistance-increasing section configured between the blood sample entrance and the first end; and
a slow flow section configured between the blood sample exit and the second end,
wherein the resistance-increasing section and the slow flow section decrease the flow rate of the blood sample in the bead mooring section.

7. The microchannel system as claimed in claim 1, further comprising a treated sample area connected to the blood sample exit or the pump, wherein the treated sample area recovers the treated blood sample.

8. The microchannel system as claimed in claim 1, wherein the pump is an air extracting pump, a vacuum pump, or a peristaltic pump.

9. A microchannel structure for removing circulating tumor cells in a blood without damaging cells in the blood, wherein the microchannel is loaded with a plurality of beads, and comprises:
a blood sample entrance passing a blood sample therethrough;
a bead mooring section including:
a first end connected to the blood sample entrance;
a second end;
a first section being relatively close to the first end, and cooperating with the first end to cause the plurality of beads to form a bead array in the bead mooring section for decreasing a flow rate of the blood sample through an interstice among neighboring ones of the plurality of beads; and
a second section being relatively close to the second end, and causing the treated blood sample to smoothly flow therethrough; and
a blood sample exit connected to the second end.

10. The microchannel structure as claimed in claim 9, wherein the first section is a bead blocking wall built in the bead mooring section, the first end has a curvy structure, the plurality of beads are blocked by the bead blocking wall and form the bead array between the bead blocking wall and the first end, and the blood sample is treated by the bead array.

11. The microchannel structure as claimed in claim 10, wherein the second section has a centrally protruding structure connected to the bead blocking wall, the two sides of the centrally protruding structure form two channels with the second end, and the two channels cause the treated blood sample to smoothly flow therethrough.

12. The microchannel structure as claimed in claim 11, wherein the centrally protruding structure is a stepped part.

13. The microchannel structure as claimed in claim 11, wherein each of the plurality of beads has a particle size, each of the two channels has an aperture, and the particle size is larger than the aperture to prevent the plurality of beads from entering into the two channels.

14. The microchannel structure as claimed in claim 9, further comprising a resistance-increasing section configured between the blood sample entrance and the first end, wherein a width of the resistance-increasing section is smaller than that of the bead mooring section, so as to decrease the flow rate of the blood sample in the bead mooring section.

15. The microchannel structure as claimed in claim 9, further comprising a slow flow section having a first aperture and configured between the blood sample exit and the second end, wherein the slow flow section is a labyrinth structure, the bead mooring section has a second aperture, and the first aperture is smaller than the second aperture so as to decrease the flow rate of the blood sample in the bead mooring section.

16. A method for removing circulating tumor cells in a blood without damaging cells in the blood, comprising steps of:
(a) providing a microchannel structure mooring therein a plurality of beads formed as a bead array to decrease therein a flow rate of the blood, wherein each of the plurality of beads has a surface coated therewith a plurality of antibodies;
(b) obtaining a blood sample from a subject;
(c) causing the blood sample to flow through the microchannel structure; and
(d) catching the circulating tumor cells in the blood sample by the plurality of antibodies.

17. The method as claimed in claim 16, further comprising steps of:
(e) recovering the treated blood sample, wherein an amount of the circulating tumor cells in the treated blood sample is less than that in the untreated blood sample; and
(f) transfusing the treated blood sample back to the subject.

18. The method as claimed in claim 17, further comprising a step of:
(g) repeating step (a) to step (f) until there is no circulating tumor cells in the subject.

19. The method as claimed in claim 16, further comprising steps of:
(e) recovering the treated blood sample;
(f) determining whether there still are the circulating tumor cells in the treated blood sample; and
(g)transfusing the treated blood to another subject in need thereof if no circulating tumor cells are detected.

20. The method as claimed in claim 16, further comprising a step of:
(e) analyzing the circulating tumor cells caught by the plurality of beads.
